(19) United States
(12) Patent Application Publication
(10) Pub. No.: US 2010/0068226 A1
(43) Pub. Date: Mar. 18, 2010

(54) Polynucleotides and Uses Thereof

(76) Inventors: Geraldine Taylor, Berkshire (GB); Gary Bembridge, Herts (GB)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/523,342

(22) PCT Filed: Jan. 18, 2008
(86) PCT No.: PCT/GB2008/000178

§ 371 Date: Nov. 30, 2009

(30) Foreign Application Priority Data
Jan. 18, 2007 (GB) 0700914.5

(51) Int. Cl. A61K 39/155; C07H 21/04; C12N 15/74; C12N 5/071; C12N 5/10; C12N 5/07; C12N 15/09; A61K 31/7088; A61P 31/12; A61P 37/04

(52) U.S. Cl. 424/211.1; 536/23.72; 435/320.1; 435/369; 435/358; 435/354; 435/364; 435/69.3; 514/44 R

(57) ABSTRACT

The present invention provides an isolated polynucleotide comprising or consisting of the nucleotide sequence encoding the G protein of human respiratory syncytial virus (RSV), wherein the nucleotide sequence is codon optimised for expression in mammalian cells and wherein the polynucleotide provides increased expression of the G protein in mammalian cells relative to expression of the wildtype RSV-G gene. Preferably, the polynucleotide comprises or consists of the nucleotide sequence of SEQ ID NO:2. Further aspects of the invention provide pharmaceutical compositions, in particular vaccines, for use in methods of immunising a subject against RSV infection.

### FIELD OF INVENTION

The present invention relates to polynucleotide molecules encoding the G protein of human respiratory syncytial virus (RSV) and the use thereof in vaccine compositions.

### INTRODUCTION

Respiratory syncytial virus (RSV) is the most common cause of acute respiratory illness in the paediatric population (see Anderson, 2000, Vaccine 19(Suppl 1): S59-65). Up to 2% of children are hospitalised as a result of serious RSV infection during their first year of life and most children will have been infected by their second year of life. RSV infections also occur within the adult population and seasonal community outbreaks commonly occur, predominantly during winter months in temperate climates, or during the rainy season in tropical climates. Immunocompromised patients, those with underlying lung or heart disease and the elderly are particularly susceptible to severe infection (see Brandenburg et al., 2001 Vaccine 19 (20-22):2769-82). For these reasons, RSV is a high priority for vaccine development.

Since the peak incidence of severe RSV infection occurs between 2 and 7 months of age, vaccination is required during the first month of life. However, the immunosuppressive effects of maternal antibody and the relative immaturity of the immune system constitute major obstacles to successful vaccination at this time (see Siegrist et al., 1999, J Infect Dis 179(6):1326-33). Natural infection with RSV does not induce long-term immunity, although severity of disease decreases with subsequent infections. Ideally, any vaccination strategy should be more effective at inducing protection than natural infection. In addition to these obstacles, previous efforts to develop a vaccine using a formalin-inactivated virus have actually resulted in an enhancement of pulmonary pathology following RSV infection (see Collins & Murphy, 2002, Virology 296(2):204-11).

The F and G glycoproteins are the major protective antigens of RSV and are the only proteins that induce neutralising antibodies and long-term protective immunity, and so are of most interest for vaccine development (see Anderson, 2000, Vaccine 19(Suppl 1):S59-65; Collins, P. L., Charnock & Murphy, Virology: Respiratory Syncytial Virus. 4th ed. Fields' Virology, ed. D. M. a. H. Knipe, P. M. 2001, Philadelphia: Lippincot Williams & Wilkins. 1443-1485; Connors et al., 1991, J Virol 65(3):1634-7). The F protein mediates virus entry, resulting from fusion of the virion envelope with the host cell plasma membrane. Later in infection the F protein is expressed on the cell surface and mediates fusion with neighbouring cells to produce multinucleated giant cells. The F protein is conserved between strains of virus and can induce protection against both RSV A and B subgroups (see Johnson et al., 1987, J Virol 61(10):3163-6).

The RSV G protein is one of the main attachment proteins. It is a type II transmembrane glycoprotein, anchored at the amino terminus. About 50% of the protein produced is secreted from the cell because of an alternative initiation codon in the transmembrane domain. The G protein is highly glycosylated with 60% of its mass due to carbohydrate. There is variation in the G protein between and within each RSV subgroup, so the G protein confers only sub-group specific protection (see Johnson et al., 1987, J Virol 61(10):3163-6).

DNA vaccination, which involves the delivery of a plasmid encoding antigen, elicits both humoral and cellular immune responses, and engages both the MHC class I and MHC class II antigen-processing pathways. This allows priming of both CD8⁺ and CD4⁺T cells (see Leitner et al., 1999, Vaccine 18(9-10):765-77). DNA vaccines are relatively simple and inexpensive to produce, with a gene cloned into a commercially available plasmid within a matter of weeks, making it a more cost effective method than producing recombinant viruses (see Hasan et al., 1999, J Immunol Methods 229(1-2):1-22; Doria-Rose & Haigwood, 2003, Methods 31(3):207-16). Plasmid vectors are highly temperature stable, making them ideal for developing countries where the cold chain is difficult to maintain, as well as offering a safe and viable alternative to recombinant vector constructs, eliminating both the problem of the pathogenic potential of the recombinant vector and the possibility of an immune response to the vector rather than the encoded gene (see Hasan et al., 1999, J Immunol Methods 229(1-2):1-22). DNA vaccines can also encode multiple antigens, without any issues of incompatible buffers or stabilisers (see Doria-Rose & Haigwood, 2003, Methods 31(3):207-16). Furthermore, there is evidence that DNA vaccination can overcome the suppressive effects of maternal antibody (see Martinez et al., 1999, Eur J Immunol 29(10):3390-400).

In many cases, however, DNA vaccines are hampered by poor efficacy (see Leitner et al., 1999, Vaccine 18(9-10):765-77). Expression levels may be low due to instability of the secondary structure or transport of the mRNA, negative regulatory sequences, the use of rare codons, or the antigen being expressed may be toxic to the target cells.

The present invention seeks to provide agents for use in the preparation of effective vaccines against RSV infection.

The listing or discussion of an apparently prior-published document in this specification should not necessarily be taken as an acknowledgement that the document is part of the state of the art or is common general knowledge.

### SUMMARY OF INVENTION

A first aspect of the invention provides an isolated polynucleotide comprising or consisting of the nucleotide sequence encoding a G protein of human respiratory syncytial virus (RSV), wherein the nucleotide sequence is codon optimised for expression in mammalian cells and wherein the polynucleotide provides increased expression of the G protein in mammalian cells relative to expression of the wildtype RSV-G gene.

By a “G protein” of RSV we include a polypeptide comprising or consisting of the amino acid sequence of SEQ ID NO:1 (see NCBI accession number AAB59857), as well as immunogenic fragments and variants of the same.

| [SEQ ID NO: 1] | |
|---|---|
| 1 | MSKNKDQRTA KTLERTWDTL NHLLFISSCL YKLNLKSVAQ ITLSILAMII STSLIIAAII |
| 61 | FIASANHKVT PTTAIIQDAT SQIKNTTPTY LTQNPQLGIS PSNPSEITSQ ITTILASTTP |
| 121 | GVKSTLQSTT VKTKNTTTTQ TQPSKPTTKQ RQNKPPSKPN NDFHFEVFNF VPCSICSNNP |
| 181 | TCWAICKRIP NKKPGKKTTT KPTKKPTLKT TKKDPKPQTT KSKEVPTTKP TEEPTINTTK |
| 241 | TNIITTLLTS NTTGNPELTS QMETFHSTSS EGNPSPSQVS TTSEYPSQPS SPPNTPRQ |

The amino acid sequence of G proteins of RSV is also disclosed in the following database entries; AAS79119, AAS79140, AAS79139, AAS79138, AAS79137, AAS79136, AAS79135, AAS79134, AAS79133, AAS79132, AAS79131, AAS79130, AAS79129, AAS79128, AAS79127, AAS79126, AAS79125, AAS79124, AAS79123 and AAS79122.

It will be appreciated by persons skilled in the art that the polynucleotide molecule of the first aspect of the invention may comprise or consist of DNA, RNA and synthetic oligonucleotides, as well as analogues, conjugates and derivatives thereof. Such nucleic acid molecules may be double-stranded or single-stranded. Preferably, however, the polynucleotide molecule is a DNA molecule.

Polynucleotide molecules of the invention may be made by methods well known to persons skilled in the art (see Sambrook & Russell, 2001, Molecular Cloning: A Laboratory Manual, Third Edition, Cold Spring Harbor Press, New York). For example, the nucleic acid molecules may be synthesised chemically or produced using a cloning vector.

By “isolated” we mean that the polynucleotide is provided in a form distinct from that in which it is found in nature. In a preferred embodiment, the polynucleotide is a recombinant polynucleotide which may be inserted into a suitable expression vector and expressed in a suitable mammalian host cell (see below).

It will be appreciated by persons skilled in the art that each amino acid making up a gene sequence can be encoded by several different codons. The use of particular codons for any amino acid is not random in most cases and appears to be species specific, with each species showing a particular codon bias (see Sharp & Li, 1987, Nucleic Acids Res 15(3):1281-95). When preparing a DNA vaccine it is possible to produce a synthetic sequence by substituting wild-type codons for ones that are optimised for the organism in which the gene is to be expressed, thereby increasing gene expression in the host.

Thus, by “codon optimised” we mean that the polynucleotide molecule of the first aspect of the invention comprises or consists of a nucleotide sequence wherein some or all of the codons present in the wildtype G gene sequence are replaced with codons which encode the same amino acid but are more commonly found in mammalian genes. Preferably, the codons are optimised for expression of the G protein in human cells.

Methods of codon optimisation are well known in the art, for example the Upgene and Codon Optimizer software, as described in Gao et al., 2004, Biotechnol Prog. 20(2):443-8 and Fuglsang, 2003, Protein Expr Purif. 31(2):247-9. Alternative codon optimisation resources include JCat (see Nucleic Acid Research, 2005, 33:W526-531) and Synthetic Gene Designer (see www.evolvingcode.net/codon/sgd/index.php)

By “increased expression” we include that G protein production by expression of the codon optimised polynucleotide of the invention in a mammalian host cell is at least 10% greater than G protein production by expression of the wildtype G gene in the same host cell. Preferably, expression is increased by at least 20%, 30%, 40%, 50%, 75%, 100%, 200%, 300%, 400%, 500%, 600%, 700%, 800%, 900% or 1000% or more.

In a preferred embodiment of the first aspect of the invention, the polynucleotide encoding the G protein of RSV is codon optimised for increased expression in human cells, such as HEK 293 cells.

Also included within the scope of the present invention are codon optimised polynucleotides encoding immunogenic fragments and variants of a full-length G protein.

By “immunogenic” we mean that the fragment or variant is capable of inducing an immune response, i.e. production of anti-G protein antibodies, when administered to a subject. Preferably, the subject is human.

By “fragment” we mean that the polynucleotide of the invention encodes an incomplete portion of a naturally occurring G protein. Preferably, the polynucleotide encodes at least 10 contiguous amino acids of a naturally-occurring (wildtype) G protein from RSV, more preferably at least 12, 14, 16, 18, 20, 22, 24, 26, 28, 30, 31, 32, 33, 34 or 35 contiguous amino acids.

By “variant” we mean that the polynucleotide of the invention encodes a non-naturally occurring G protein or fragment thereof. Thus, we include insertions, deletions and substitutions, either conservative or non-conservative, where such changes do not substantially alter the immunogenicity of the polypeptide. By conservative substitutions is intended combinations such as Gly, Ala; Val, Ile, Leu; Asp, Glu; Asn, Gln; Ser, Thr; Lys, Arg; and Phe, Tyr. Such variants may be made using the well-known methods of protein engineering and site-directed mutagenesis. Preferably, the polynucleotide encodes a polypeptide which shares at least 60% amino acid sequence identity to a naturally-occurring (wildtype) G protein from RSV, more preferably at least 70% or 80% or 85% or 90% identity to said sequence, and more preferably at least 95%, 96%, 97%, 98% or 99% identity to a wildtype G protein.

The percent sequence identity between two polypeptides may be determined using suitable computer programs, for example the GAP program of the University of Wisconsin Genetic Computing Group and it will be appreciated that percent identity is calculated in relation to polypeptides whose sequences have been aligned optimally.

The alignment may alternatively be carried out using the Clustal W program (Thompson et al (1994) Nucl Acid Res 22, 4673-4680). The parameters used may be as follows:

Fast pairwise alignment parameters: K-tuple(word) size; 1, window size; 5, gap penalty; 3, number of top diagonals; 5. Scoring method: x percent.

Multiple alignment parameters: gap open penalty; 10, gap extension penalty; 0.05.

Scoring matrix: BLOSUM.

Methods of producing a polynucleotide of the first aspect of the invention are well known to those skilled in the art. Examples of such methods are discussed in Sambrook et al., Molecular Cloning: A Laboratory Manual, 2001, 3rd edition and also discussed in Examples below.

In a preferred embodiment of the first aspect of the invention, the polynucleotide comprising or consisting of the nucleotide sequence of SEQ ID NO:2.

| | |
|---|---|
| | [SEQ ID NO: 2] |
| 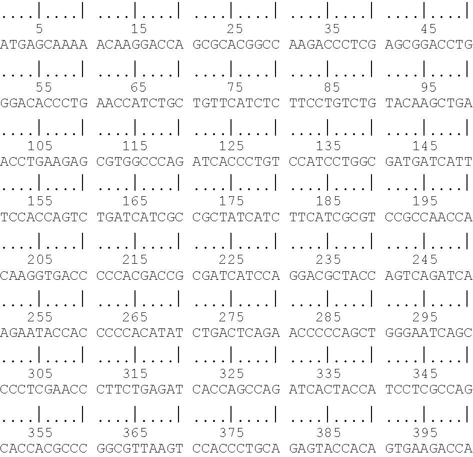 | |
| 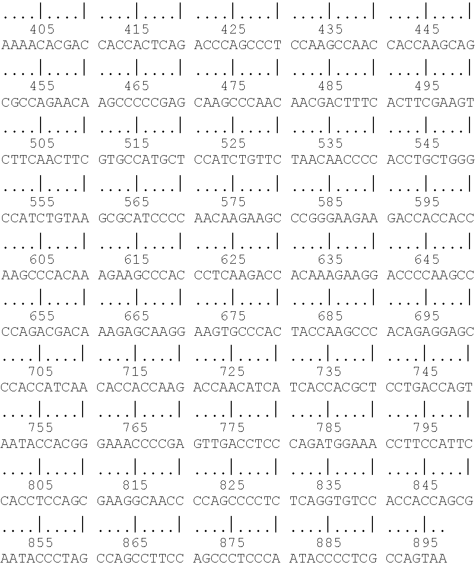 | |

More preferably, the polynucleotide consists of the nucleotide sequence of SEQ ID NO:2.

In a further embodiment, the polynucleotide may be comprised in a delivery vehicle for delivering nucleic acid to the target. The delivery vehicle may be any suitable delivery vehicle. It may, for example, be a liposome containing nucleic acid, or it may be a virus or virus-like particle which is able to deliver nucleic acid.

A polynucleotide according to the first aspect of the invention may be used in accordance with known techniques, appropriately modified in view of the teachings contained herein, to construct an expression vector.

Generally, the polynucleotide is inserted into an expression vector, such as a plasmid, in proper orientation and correct reading frame for expression. If necessary, the polynucleotide, e.g. DNA, may be linked to the appropriate transcriptional and translational regulatory control nucleotide sequences recognised by the desired host, although such controls are generally available in the expression vector. Thus, the polynucleotide insert may be operatively linked to an appropriate promoter. Eukaryotic promoters include the CMV immediate early promoter, the HSV thymidine kinase promoter, the early and late SV40 promoters and the promoters of retroviral LTRs. Other suitable promoters will be known to the skilled artisan. The expression constructs will desirably also contain sites for transcription initiation and termination, and in the transcribed region, a ribosome-binding site for translation. (see WO 98/16643).

Suitable eukaryotic expression vectors include animal cell systems transfected with, for example, adenovirus expression vectors. Examples of such vectors include pI, pSI and pCI mammalian expression vectors (Promega Corp). A typical mammalian cell vector plasmid is pSVL available from Pharmacia (Piscataway, N.J., USA). This vector uses the SV40 late promoter to drive expression of cloned genes, the highest level of expression being found in T antigen-producing cells, such as COS-1 cells. An example of an inducible mammalian expression vector is pMSG, also available from Pharmacia (Piscataway, N.J., USA). This vector uses the glucocorticoid-inducible promoter of the mouse mammary tumour virus long terminal repeat to drive expression of the cloned gene.

Methods well known to those skilled in the art can be used to construct expression vectors containing the coding sequence and, for example appropriate transcriptional or translational controls. One such method involves ligation via homopolymer tails. Homopolymer polydA (or polydC) tails are added to exposed 3′ OH groups on the DNA fragment to be cloned by terminal deoxynucleotidyl transferases. The fragment is then capable of annealing to the polydT (or polydG) tails added to the ends of a linearised plasmid vector. Gaps left following annealing can be filled by DNA polymerase and the free ends joined by DNA ligase.

Another method involves ligation via cohesive ends. Compatible cohesive ends can be generated on the DNA fragment and vector by the action of suitable restriction enzymes. These ends will rapidly anneal through complementary base pairing and remaining nicks can be closed by the action of DNA ligase.

A further method uses synthetic molecules called linkers and adaptors. DNA fragments with blunt ends are generated by bacteriophage T4 DNA polymerase or E. coli DNA polymerase I which remove protruding 3′ termini and fill in recessed 3′ ends. Synthetic linkers, pieces of blunt-ended double-stranded DNA which contain recognition sequences for defined restriction enzymes, can be ligated to blunt-ended DNA fragments by T4 DNA ligase. They are subsequently digested with appropriate restriction enzymes to create cohesive ends and ligated to an expression vector with compatible termini. Adaptors are also chemically synthesised DNA fragments which contain one blunt end used for ligation but which also possess one preformed cohesive end.

Synthetic linkers containing a variety of restriction endonuclease sites are commercially available from a number of sources including International Biotechnologies Inc, New Haven, Conn., USA.

A desirable way to modify the DNA encoding the polypeptide of the invention is to use the polymerase chain reaction as disclosed by Saiki et al. (1988) Science 239, 487-491. In this method the DNA to be enzymatically amplified is flanked by two specific oligonucleotide primers which themselves become incorporated into the amplified DNA. The said specific primers may contain restriction endonuclease recognition sites which can be used for cloning into expression vectors using methods known in the art.

Thus, a second aspect of the invention provides a vector comprising a polynucleotide according to the first aspect of the invention. Preferably, the vector is an expression vector. For example, the vector may be selected from the group consisting of other paramyxoviruses such as human parainfluenza virus type 3 (PIV3), bovine PIV3, Sendai virus, Newcastle Disease virus, vaccinia virus, fowlpox virus, semliki forest virus etc.

In a particularly preferred embodiment, the vector is pI.17 (see Bembridge et al., 2000, J. Gen. Virol. 81:2519-23.

Alternatively, the vector may be RSV itself, modified to comprise a polynucleotide according to the first aspect of the invention.

A third aspect of the invention provides a mammalian host cell comprising a polynucleotide or a vector according to the first and second aspects of the invention, respectively. Preferably, the host cell is selected from the group consisting of human embryonic kidney cells (for example HEK293 and HEL203 cells), Chinese hamster ovary cells (or CHO cells, for example available from the ATCC as CCL61), NIH Swiss mouse embryo cells NIH/3T3 (for example, available from the ATCC as CRL 1658), and monkey kidney-derived COS-1 cells (for example, available from the ATCC as CRL 1650).

More preferably, the host cell is a human cell or derived from such a cell. In a particularly preferred embodiment, the host cell is an HEK 293 cell.

A fourth aspect of the invention provides the use of a polynucleotide, a vector or a host cell according to the first, second or third aspects of the invention, respectively, in the production of a G protein, or immunogenic fragment or variant thereof. The invention thus provides a method for producing a polypeptide according to SEQ ID NO:1, or immunogenic fragment or variant thereof, the method comprising expressing a polynucleotide according to the first aspect of the invention in a host cell, and isolating the expressed polypeptide therefrom.

Advantageously, the resultant polypeptide is substantially free of other RSV polypeptides and substantially free of host cell components.

The method may make use of conventional protein purification strategies such as size exclusion chromatography or ion-exchange chromatography.

In one embodiment, the method further comprises admixing the expressed polypeptide with a pharmaceutically acceptable excipient, diluent or carrier to produce a pharmaceutical composition (such as a vaccine composition).

Thus, the invention provides a method of producing a G protein, or immunogenic fragment or variant thereof, for use in vaccine compositions.

It will be appreciated by persons skilled in the art that most proteins are poorly immunogenic or non-immunogenic when administered alone. Strong adaptive immune responses to protein antigens almost always require that the antigen be injected in a mixture with an agent known as an adjuvant. An adjuvant is any substance that enhances the immunogenicity of substances mixed with it. Adjuvants differ from protein carriers in that they generally do not form stable linkages with the immunogen, although one exception to this is the adduction of reactive carbonyls to antigens. Furthermore, adjuvants are needed primarily for initial immunisations, whereas carriers are required to elicit not only primary but also subsequent responses to haptens. Commonly used adjuvants include imiquimod, Freund's (complete and incomplete), mineral gels (e.g. aluminium hydroxide), surface-active substances (e.g. lysolecithin, pluronic polyols, polyanions, peptides, oil emulsions, dinitrophenol, etc.), adjuvants usable in humans such as Bacille Calmette-Guérin and Corynebacterium parvum, or similar immunostimulatory agents. Additional examples of adjuvants that can be employed include MPL-TDM adjuvant (monophosphoryl Lipid A, synthetic trehalose dicorynomycolate). For example, see “Vaccine adjuvants” 2000, Ed. Derek O′Hagan, Humana Press, New Jersey.

Adjuvants can enhance immunogenicity in several different ways. First, adjuvants convert soluble protein antigens into particulate material, which is more readily ingested by antigen-presenting cells such as macrophages. For example, the antigen can be adsorbed on particles of the adjuvant (such as alum), made particulate by emulsification in mineral oils, or incorporated into the colloidal particles of ISCOMs or biodegradable synthetic beads. This enhances immunogenicity somewhat, but such adjuvants are relatively weak unless they also contain bacteria or bacterial products. Such microbial constituents are a second means by which adjuvants enhance immunogenicity, and although their exact contribution to enhancing immunogenicity is unknown, they are clearly the more important component of an adjuvant. Microbial products may signal macrophages or dendritic cells to become more effective antigen-presenting cells. One of their effects is to induce the production of inflammatory cytokines and potent local inflammatory responses; this effect is probably intrinsic to their activity in enhancing responses, but largely precludes their use in humans. A third means to achieve an adjuvant effect is to adduct a reactive carbonyl to an antigen (see above).

Persons skilled in the art will appreciate that the antigenic G protein, or fragment or variant thereof, may be fused to another moiety, such as a polypeptide. Typically, the polypeptide is one which is able to enhance the immune response to the polypeptide to which it is fused. The fusion partner may also be a polypeptide that facilitates purification, for example by constituting a binding site for a moiety that can be immobilised in, for example, an affinity chromatography column. Thus, the fusion partner may comprise oligo-histidine or other amino acids which bind to cobalt or nickel ions. It may also be an epitope for a monoclonal antibody such as a Myc epitope.

Whilst it is possible for an antigenic polypeptide to be administered alone, it is preferable to present it as a pharmaceutical formulation, together with one or more acceptable carriers. The carrier(s) must be “acceptable” in the sense of being compatible with the said antigen and not deleterious to the recipients thereof. Typically, the carriers will be water or saline which will be sterile and pyrogen free.

The formulations may conveniently be presented in unit dosage form and may be prepared by any of the methods well known in the art of pharmacy. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

It will be appreciated by persons skilled in the art that the polynucleotides of the first aspect of the invention may also be used directly in vaccine preparations, e.g. as DNA vaccines (for example, see Ivory & Chadee, 2004, Genetic Vaccines & Therapy 2:17). Codon-optimisation of the wildtype G gene provides DNA vaccines capable of increased expression of the antigenic protein, and hence increased efficacy.

Thus, a fifth aspect of the present invention provides a pharmaceutical composition comprising a polynucleotide, a vector or a host cell according to the first, second or third aspects of the invention, respectively, and a pharmaceutically acceptable excipient, diluent or carrier. Preferably, the composition is a vaccine composition.

By “pharmaceutically acceptable” is included that the formulation is sterile and pyrogen free. Suitable pharmaceutical carriers are well known in the art of pharmacy. The carrier(s) must be “acceptable” in the sense of being compatible with the compound of the invention and not deleterious to the recipients thereof. Typically, the carriers will be water or saline which will be sterile and pyrogen free; however, other acceptable carriers may be used. Thus, “pharmaceutically acceptable” excipient, diluent or carrier includes any compound(s) used in forming a part of the formulation that is intended to act merely as a carrier, i.e. not intended to have biological activity itself. The pharmaceutically acceptable excipient, diluent or carrier is generally safe, non-toxic, and neither biologically nor otherwise undesirable. A pharmaceutically acceptable excipient, diluent or carrier as used herein includes both one and more than one such excipient, diluent or carrier.

The polynucleotide or expression vector is preferably purified from the host cell in which it is produced (or if produced by peptide or polynucleotide synthesis, purified from any contaminants of the synthesis). Typically, the polynucleotide or expression vector contains less than 5% of contaminating material, preferably less than 2%, 1%, 0.5%, 0.1%, 0.01%, before it is formulated for use in medicine, for example for use in a vaccine. The polynucleotide or expression vector desirably is substantially pyrogen free.

It will be appreciated by persons skilled in the art that the G protein derived polypeptides or polynucleotides described herein will generally be administered in admixture with a suitable pharmaceutical excipient diluent or carrier selected with regard to the intended route of administration and standard pharmaceutical practice (for example, see Remington: The Science and Practice of Pharmacy, 19^{th} edition, 1995, Ed. Alfonso Gennaro, Mack Publishing Company, Pennsylvania, USA).

For example, the pharmaceutical compositions can be administered orally, buccally or sublingually in the form of tablets, capsules, ovules, elixirs, solutions or suspensions, which may contain flavouring or colouring agents, for immediate-, delayed- or controlled-release applications.

Typically, the pharmaceutical (e.g. vaccine) compositions of the present invention will be administered parenterally, for example, intravenously, intraperitoneally, intranasally, intra-muscularly, intra-dermally, subcutaneously, or they may be administered by infusion techniques. They are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Where the active ingredient is a polypeptide (i.e. the G protein or fragment or variant thereof), it may be preferable to use a sustained-release drug delivery system, such as a microsphere. These are designed specifically to reduce the frequency of injections. An example of such a system is Nutropin Depot which encapsulates recombinant human growth hormone (rhGH) in biodegradable microspheres that, once injected, release rhGH slowly over a sustained period.

Alternatively, polypeptide medicaments and agents can be administered by a surgically implanted device that releases the agent directly at the required site.

Electroporation therapy (EPT) systems can also be employed for the administration of proteins, polypeptides and DNA vaccines. A device which delivers a pulsed electric field to cells increases the permeability of the cell membranes to the drug, resulting in a significant enhancement of intracellular drug delivery.

Proteins and polypeptides can also be delivered by electroincorporation (EI). EI occurs when small particles of up to 30 microns in diameter on the surface of the skin experience electrical pulses identical or similar to those used in electroporation. In EI, these particles are driven through the stratum corneum and into deeper layers of the skin. The particles can be loaded or coated with drugs or genes or can simply act as “bullets” that generate pores in the skin through which the drugs can enter.

An alternative method of protein and polypeptide delivery is the thermo-sensitive ReGel injectable. Below body temperature, ReGel is an injectable liquid while at body temperature it immediately forms a gel reservoir that slowly erodes and dissolves into known, safe, biodegradable polymers. The active drug is delivered over time as the biopolymers dissolve.

Protein and polypeptide pharmaceuticals can also be delivered orally. One such system employs a natural process for oral uptake of vitamin B12 in the body to co-deliver proteins and polypeptides. By riding the vitamin B12 uptake system, the protein or polypeptide can move through the intestinal wall. Complexes are produced between vitamin B12 analogues and the drug that retain both significant affinity for intrinsic factor (IF) in the vitamin B12 portion of the complex and significant bioactivity of the drug portion of the complex

Methods for administering oligonucleotide or polynucleotide active agents (i.e. a polynucleotide of the first aspect of the invention) are also well know in the art (see Dass, 2002, J Pharm Pharmacol. 54(1):3-27; Dass, 2001, Drug Deliv. 8(4):191-213; Lebedeva et al., 2000, Eur J Pharm Biopharm. 50(1):101-19; Pierce et al., 2005, Mini Rev Med. Chem. 5(1):41-55; Lysik & Wu-Pong, 2003, J Pharm Sci. 2003 2(8):1559-73; Dass, 2004, Biotechnol Appl Biochem. 40(Pt 2):113-22; Medina, 2004, Curr Pharm Des. 10(24):2981-9.

DNA-based vaccine compositions are relatively simple and inexpensive to produce, with a gene cloned into a commercially available plasmid within a matter of weeks, making it a more cost effective method than producing recombinant viruses (for example, see Hasan et al., 1999, J Immunol Methods 229(1-2):1-22; Doria-Rose & Haigwood, 2003, Methods, 31(3):207-16). Plasmid vectors are highly temperature stable, making them ideal for developing countries where the cold chain is difficult to maintain, as well as offering a safe and viable alternative to recombinant vector constructs, eliminating both the problem of the pathogenic potential of the recombinant vector and the possibility of an immune response to the vector rather than the encoded gene (see Hasan et al., 1999, J Immunol Methods 229(1-2):1-22). DNA vaccines can also encode multiple antigens, without any issues of incompatible buffers or stabilisers (Doria-Rose & Haigwood, 2003, Methods, 31(3):207-16). Furthermore, there is evidence that DNA vaccination against RSV can overcome the suppressive effects of maternal antibody (see Martinez et al., 1999, Eur J Immunol 29(10):3390-400).

Other methods involve simple delivery of the construct into the cell for expression therein. An example of such an approach includes liposomes (Nassander et al (1992) Cancer Res. 52, 646-653).

For the preparation of immuno-liposomes MPB-PE (N-[4-(p-maleimidophenyl)butyryl]-phosphatidylethanolamine) is synthesised according to the method of Martin & Papahadjopoulos (1982) J. Biol. Chem. 257, 286-288. MPB-PE is incorporated into the liposomal bilayers to allow a covalent coupling of the antibody, or fragment thereof, to the liposomal surface. The liposome is conveniently loaded with the agent of the invention (such as DNA or other genetic construct) for delivery to the target cells, for example, by forming the said liposomes in a solution of the agent, followed by sequential extrusion through polycarbonate membrane filters with 0.6 μtin and 0.2 μm pore size under nitrogen pressures up to 0.8 MPa. After extrusion, entrapped DNA construct is separated from free DNA construct by ultracentrifugation at 80 000×g for 45 min. Freshly prepared MPB-PE-liposomes in deoxygenated buffer are mixed with freshly prepared antibody (or fragment thereof) and the coupling reactions are carried out in a nitrogen atmosphere at 4° C. under constant end over end rotation overnight.

The immunoliposomes are separated from unconjugated antibodies by ultracentrifugation at 80 000×g for 45 min. Immunoliposomes may be injected intraperitoneally or directly into the tumour.

Other methods of delivery include adenoviruses carrying external DNA via an antibody-polylysine bridge (see Curiel Prog. Med. Virol. 40, 1-18) and transferrin-polycation conjugates as carriers (Wagner et al (1990) Proc. Natl. Acad. Sci. USA 87, 3410-3414). In the first of these methods a polycation-antibody complex is formed with an oligonucleotide agent of the invention, wherein the antibody is specific for either wild-type adenovirus or a variant adenovirus in which a new epitope has been introduced which binds the antibody. The polycation moiety binds the oligonucleotide agent via electrostatic interactions with the phosphate backbone. The adenovirus, because it contains unaltered fibre and penton proteins, is internalised into the cell and carries into the cell with it the oligonucleotide agent of the invention. It is preferred if the polycation is polylysine.

The oligonucleotide agent may also be delivered by adenovirus wherein it is present within the adenovirus particle, for example, as described below.

In an alternative method, a high-efficiency nucleic acid delivery system that uses receptor-mediated endocytosis to carry DNA macromolecules into cells is employed. This is accomplished by conjugating the iron-transport protein transferrin to polycations that bind nucleic acids. Human transferrin, or the chicken homologue conalbumin, or combinations thereof is covalently linked to the small DNA-binding protein protamine or to polylysines of various sizes through a disulfide linkage. These modified transferrin molecules maintain their ability to bind their cognate receptor and to mediate efficient iron transport into the cell. The transferrin-polycation molecules form electrophoretically stable complexes with DNA constructs or other genetic constructs of the invention independent of nucleic acid size (from short oligonucleotides to DNA of 21 kilobase pairs). When complexes of transferrin-polycation and the DNA constructs or other genetic constructs of the invention are supplied to the tumour cells, a high level of expression from the construct in the cells is expected.

High-efficiency receptor-mediated delivery of the DNA constructs or other genetic constructs of the invention using the endosome-disruption activity of defective or chemically inactivated adenovirus particles produced by the methods of Cotten et al (1992) Proc. Natl. Acad. Sci. USA 89, 6094-6098 may also be used. This approach appears to rely on the fact that adenoviruses are adapted to allow release of their DNA from an endosome without passage through the lysosome, and in the presence of, for example transferrin linked to the DNA construct or other genetic construct of the invention, the construct is taken up by the cell by the same route as the adenovirus particle.

This approach has the advantages that there is no need to use complex retroviral constructs; there is no permanent modification of the genome as occurs with retroviral infection; and the targeted expression system is coupled with a targeted delivery system, thus reducing toxicity to other cell types.

It will be appreciated that “naked DNA” and DNA complexed with cationic and neutral lipids may also be useful in introducing the DNA of the invention into cells of the individual to be treated. Non-viral approaches to gene therapy are described in Ledley (1995) Human Gene Therapy 6, 1129-1144.

Alternative targeted delivery systems are also known such as the modified adenovirus system described in WO 94/10323 wherein, typically, the DNA is carried within the adenovirus, or adenovirus-like, particle. Michael et al (1995) Gene Therapy 2, 660-668 describes modification of adenovirus to add a cell-selective moiety into a fibre protein. Thus, it will be appreciated that a further aspect of the invention provides a virus or virus-like particle comprising a genetic construct of the invention. Other suitable viruses or virus-like particles include RSV, HSV, AAV, vaccinia and parvovirus.

A further alternative route of DNA vaccine delivery is particle-mediated intradermal delivery (PMID) using the gene gun. The “gene gun” propels gold particles coated with DNA into the skin and deposits them within the cytoplasm of epidermal cells (see Feltquate et al., 1997, J Immunol 158(5):2278-84). One advantage of PMID gives effective immunity with a far smaller dose of DNA than that required by needle injection, typically 100- to 1000-fold less DNA (see Leitner et al., 1997, J Immunol 159(12):6112-9; Koide et al., 2000, Jpn J Pharmacol 83(3):167-74). This makes PMID a more efficient method of delivery than intramuscular injection.

A sixth aspect of the invention provides a method for producing a pharmaceutical composition according to the fifth aspect of the invention, the method comprising admixing a polynucleotide, a vector or a host cell of the invention with a pharmaceutically acceptable excipient, diluent or carrier.

An seventh aspect of the invention provides a method of immunising a subject against infection with respiratory syncytial virus, the method comprising administering to the subject a pharmaceutical composition according to the fifth aspect of the invention. Preferably, the subject is human.

Typically, the individual subject is a child, for example less than 1 month old. The method is useful in vaccinating childhood populations. The dose and frequency is determined by the physician but typically a dose of from 25 μg to 10 mg is used.

An eighth aspect of the invention provides a polynucleotide according to the first aspect of the invention or a pharmaceutical composition according to the fifth aspect of the invention for use in medicine.

A ninth aspect of the invention provides a polynucleotide according to the first aspect of the invention or a pharmaceutical composition according to the fifth aspect of the invention in the preparation of a medicament for immunisation of a subject against respiratory syncytial virus.

Preferred aspects of the invention are described in the following non-limiting examples, with reference to the following figures:

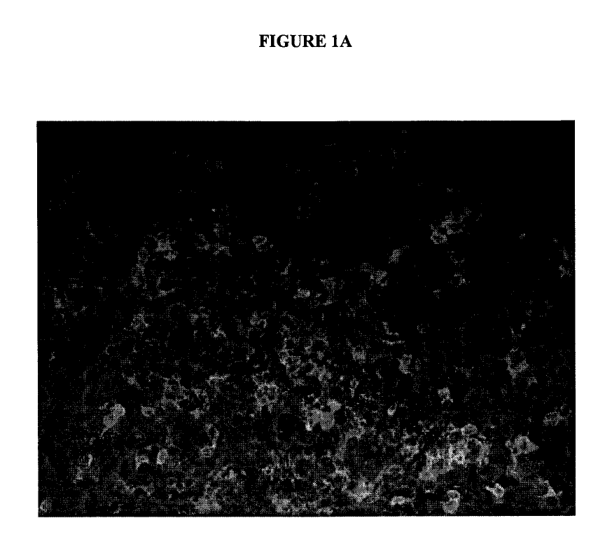

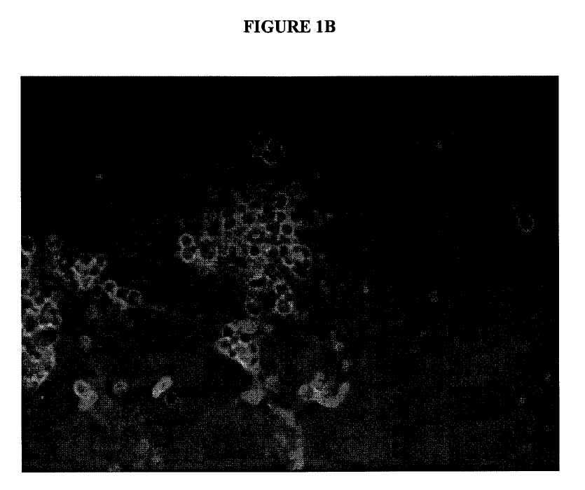

FIG. 1. Immunofluorescence of HEK 293 cells transiently transfected with pI.17Gc (a) or pI.17Gwt (b). Cells then fixed with paraformaldehyde and incubated with mAb 31, specific to RSV G protein, followed by FITC-conjugated rabbit anti-mouse IgG.

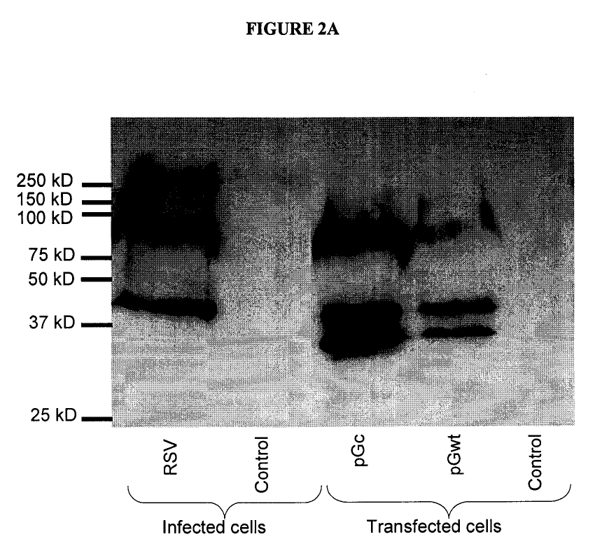

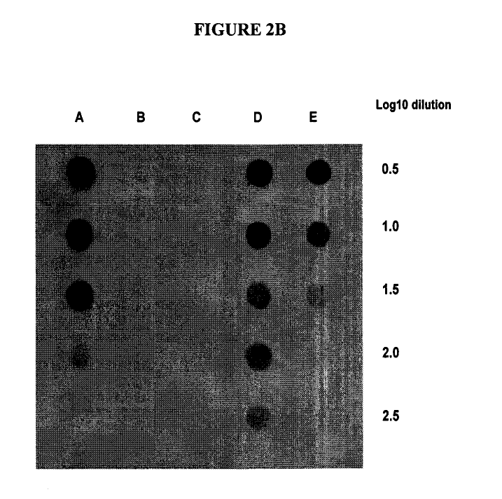

FIG. 2. Expression of the G protein in transfected COS-7 cells.

(A) Western blot analysis of expression of the G protein in transfected COS-7 cells. COS-7 cells were transfected with either pI.17Gc or pI.17Gwt. Cells were harvested after 24 h and the proteins in cell extracts were separated on a 12% polyacrylamide-SDS gel under reducing conditions and analysed by Western blotting. RSV G protein was detected using mAb 31, specific for the G protein, and HRP-conjugated goat anti-mouse IgG with SuperSignal West Pico Chemiluminescent Substrate (30 sec exposure). Controls were lysates from RSV-infected COS-7 cells, mock-infected COS-7 cells and mock-transfected COS-7 cells.

(B) Dot blot analysis of lysates from COS-7 cells transfected with either pI.17Gc (D) or pI.17Gwt (E). RSV G protein was detected using mAb 31, specific for the G protein, and HRP-conjugated goat anti-mouse IgG with SuperSignal West Pico Chemiluminescent Substrate (30 sec exposure). Controls were lysates from RSV-infected vero cells (A), mock-infected vero cells (B) and mock-infected COS-7 cells (C).

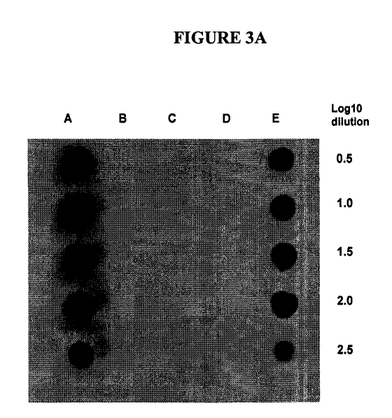

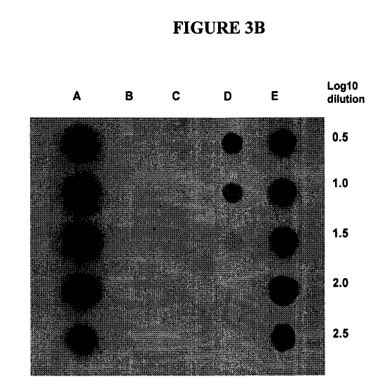

FIG. 3. Expression of the F protein in transfected COS-7 cells. Dot blot analysis of lysates from COS-7 cells transfected with either pI.17Fc (D) or pI.17Fwt (E). RSV F protein was detected using mAb 19, specific for the F protein, and HRP-conjugated goat anti-mouse IgG with SuperSignal West Pico Chemiluminescent Substrate (a) 4 min exposure, (b) 50 min exposure. Controls were lysates from RSV-infected vero cells (A), mock-infected vero cells (B) and mock-infected COS-7 cells (C).

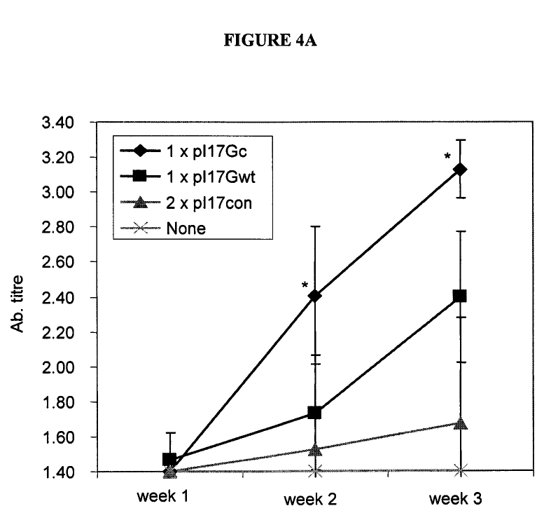

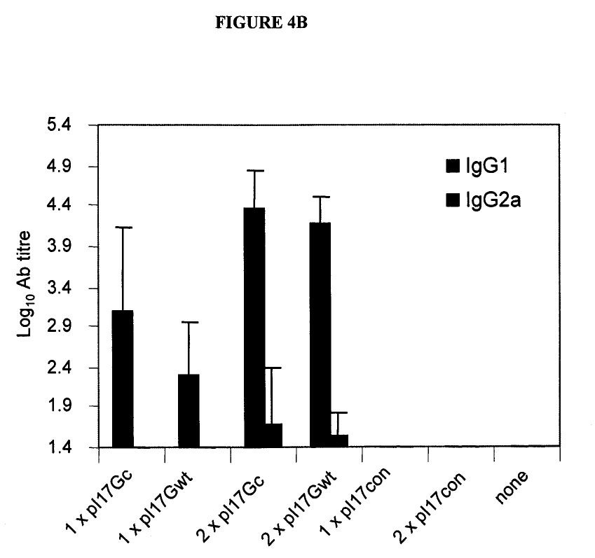

FIG. 4. RSV specific serum antibody titre.

(A) Following primary vaccination. Mice were vaccinated by gene gun with 1.5 μg of pI.17Gc, pI.17Gwt, pI.17Con or not vaccinated and test bled at weekly intervals. RSV specific antibody was analysed by ELISA. Values shown are the mean log₁₀RSV specific antibody titres (n=5). * The antibody titre from mice primed with pI.17Gc was significantly greater than that in mice vaccinated with pI.17Gwt at both 2 weeks (p<0.03) and 3 weeks (p<0.02) post vaccination.

(B) 6 weeks after vaccination. Mice were vaccinated by gene gun with 1.5 μg of either pI.17Gc, pI.17Gwt, pI.17Con or not vaccinated either once or on 2 occasions 3 weeks apart and test bled 6 weeks after vaccination. RSV specific IgG1 and IgG2a antibody were analysed by ELISA. Values shown are the mean log₁₀RSV specific IgG1 and IgG2a antibody titres (n=5). IgG1 antibody is significantly higher than IgG2a antibody in all groups primed with RSV-G plasmid (p<0.001).

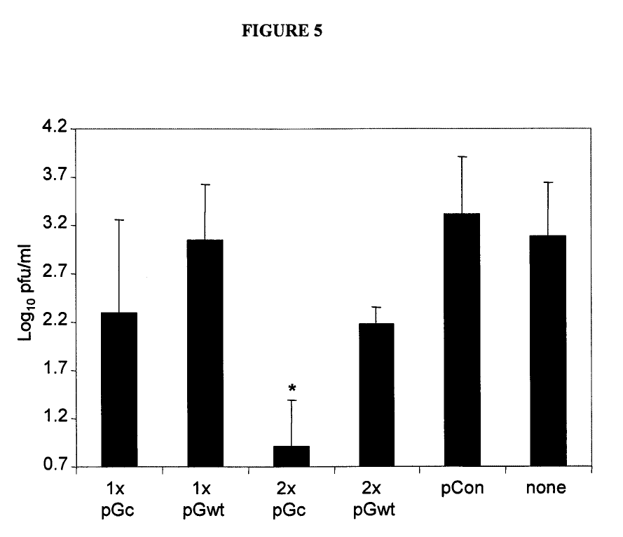

FIG. 5. RSV titres in the lungs of gene gun vaccinated mice 5 days post RSV challenge. Mice were vaccinated by gene gun with 1.5 μg of either pI.17Gc, pI.17Gwt, pI.17Con or not vaccinated either once or on 2 occasions 3 weeks apart. Mice were challenged with RSV 3 weeks after the second vaccination. Virus titres in lung homogenates were analysed by plaque assay. Values shown are the mean titre (log₁₀ pfu/ml) of RSV in lung homogenates (n=5). * The mean virus titre in mice vaccinated twice with pGc were significantly lower than in those vaccinated twice with pGwt (p<0.05).

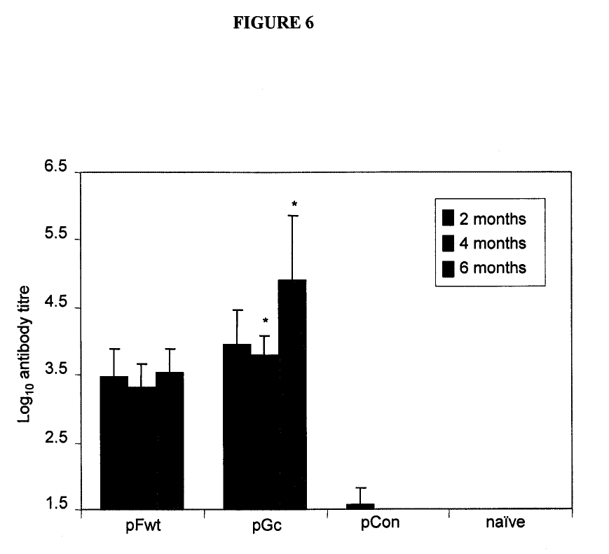

FIG. 6.

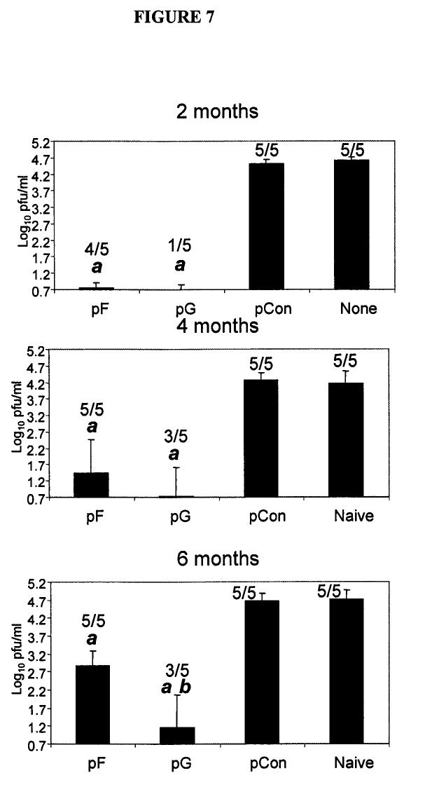

FIG. 7.

### EXAMPLES

### Example A

### Expression of Codon-Optimised F and G Sequences

### Material and Methods

### Plasmid Constructs

Using the SynGene computer program developed by Peter Ertl at GlaxoSmithkline (GSK), synthetic sequences of the F and G protein genes of RSV were generated using codons optimised for mammalian expression. Wild-type sequences for the F and G protein genes of the A2 strain of human RSV were imported into the SynGene program (Appendix 1). The mammalian codon usage co-efficient was calculated for each wild-type sequence. A codon-usage coefficient of 1 demonstrates perfect usage. Four different codon-optimized sequences were produced for F and G and imported into the Clone Manager program, which was used to map all restriction sites within each sequence. Any sequences generated by SynGene that included EcoR1 or BamH1 restriction sites within them were discarded, as these restriction sites are used to clone the gene into the expression vector. The range of unique restriction sites for commonly used restriction enzymes was also assessed for each sequence, and the sequence with the best range was selected as the final sequence. This was to aid any sequence corrections later (see Appendix 2 for codon-optimised sequences).

Using the SynGene program, each selected codon-optimised sequence had EcoR1 restriction site sequences added at the 3′ end and BamH1 restriction site sequences added at the 5′ end. SynGene was then used to generate a list of oligonucleotides that would be used to construct the synthetic genes (Appendix 3). These were all around 60 bases in length. Importing them into the Seqman program checked correct assembly of the oligonucleotides.

Oligonucleotides, produced by Invitrogen (UK), were re-hydrated to produce 100 pmol/μ1. An oligonucleotide pool was made up for the F and G genes by combining 5 μl of all of the oligonucleotides for each set. A PCR assembly reaction (Appendix 4) was run for each oligonucleotide pool and full-length products were amplified in a PCR recovery reaction (Appendix 5). The plasmid vector, pI.17, was provided by Dr J. Robertson, NIBSC, UK, and has been described previously (see Bembridge et al., 2000, J Gen Virol 81(Pt 10):2519-23). PCR products and empty pI.17 plasmid were cut with EcoR1 and BamH1 (Invitrogen, UK) in a restriction reaction (Appendix 6). Products from the restriction reactions were run on a 0.8% agarose gel and purified using a Qiagen Gel Purification kit (Qiagen, UK) following the manufacturers instructions. The purified F and G inserts were then ligated into the pI.17 plasmid (Appendix 7). The ligated plasmids containing these inserts were used to transform JM109 competent Escherichia coli cells (Promega, Madison, Wis.) following the manufacturer's instructions. Clones were grown on agar plates containing ampicillin to select for clones containing the correct insert. Clones were picked and incubated for 6 h at 37° C. in 5 ml of L broth (Becton Dickinson) to produce cultures which were then used to produce mini preps, using a Qiagen mini prep kit (Qiagen, UK) following the manufacturers instructions. Mini preps were sequenced to check for correct inserts (Appendix 8).

Corrections to the gene sequences were carried out by amplifying areas of correct sequence by PCR (Appendix 9). Amplified fragments were then stitched together by PCR using forward and reverse end primers (Appendix 10). Several rounds of corrections were required to produce clones containing the correct sequences. Giga preps of clones containing the correct sequence were produced using the Qiagen giga prep kit (Qiagen, UK) following the manufacturers instructions. These were sequenced again to confirm that they were correct.

Plasmids expressing wild-type F or G proteins were produced using forward and reverse end primers (Appendix 11) to amplify wild-type F or G genes by PCR from plasmids provided by GSK. PCR products were run on a 0.8% agarose gel and fragments were cut out and purified using the Qiagen gel purification kit (Qiagen, UK) following the manufacturers instructions. Purified products were cut with EcoR1 and BamH1 restriction enzymes and ligated into pI.17 vector (Appendix 6 and 7). The ligated plasmids containing the wild-type inserts were used to transform JM109 competent Escherichia coli cells (Promega, Madison, Wis.), grown up and used to produce mini-preps, sequenced to check for correct inserts and then grown up to produce giga-preps as described previously.

### Transient Transfections

Expression of F and G proteins was analysed by immunofluorescence of HEK 293 cells, transiently transfected with the pI.17 plasmids. In brief, HEK 293 cells were plated into a 24 well plate in Dulbecco's minimal essential medium (DMEM) (Gibco). The cells were used for transfection at 80-90% confluency. 3 μl of a mini prep of each plasmid was mixed with 50 μl of OPTI-MEM medium (Life Technology, USA) and then mixed with 6 μl Lipafectamine (Life Technology, USA) in 100 μl OPTI-MEM medium at room temperature (RT).

After 20 min incubation, the DNA-liposome complexes were added to one well and the cells were incubated at 37° C. for 24 h. Cells were then aspirated to loosen them from the wells and centrifuged at 4,600×g for 1 min. Cells were washed and re-suspended in 200 μl phosphate buffered saline (PBS).

For dot blot analysis and immunocytochemistry, COS-7 cells were transiently transfected using Superfect (Qiagen, UK) following the manufacturers instructions. Briefly, COS-7 cells were plated into 6-well plates with DMEM, 10% heat-inactivated foetal calf serum (HFCS) and 1% non-essential amino acids (Gibco). 5.5 μg plasmid DNA was added to 325 μl medium and incubated at RT for 5 min. 11 μl Superfect (Qiagen, UK) was added to each tube and incubated at RT for 10 min and then 2 ml medium was added before adding the complex to 1 well. Plates were incubated at 37° C. for 3 h, complexes were removed and fresh medium was added. Cells were incubated at 37° C. for 24 h, and analysed by immunocytochemistry. For dot blot analysis, cells were scraped off into the medium and centrifuged for 1 min at 4,600×g. The supernatant was removed, cells re-suspended in 66 μl double-deionised water (DDW) and the cell lysate stored at −20° C.

### Analysis of Protein Expression

### (i) Immunofluorescence

A volume of 3 μl of transfected HEK 293 cell suspension was dried onto glass microscope slides overnight at RT. Slides were fixed with 1% paraformaldehyde for 15 min at RT, immersed in PBS containing 0.05% tween-20 (PBS/Tw) for 2 min RT, and blocked with 10 μg/ml FITC-conjugated rabbit IgG (Dako) in PBS containing 0.1% BSA (PBS/BSA) for 30 min at RT. Appropriate monoclonal antibodies (mAb 19 (Taylor et al., 1992, J Gen Virol, 1992, 73(Pt 9):2217-23) specific for RSV F, mAb 31 (Furze and Taylor, unpublished) specific for RSV G were diluted 1/100 in PBS/BSA, and 50 μl added to each slide and incubated at 37° C. for 30 min. Slides were immersed in PBS/Tw for 5 min and 50 μl FITC-conjugated rabbit anti-mouse IgG (Nordic Immunological Laboratories, Tilburg) diluted 1/1000 was added to each slide and incubated at 37° C. for 30 min. Slides were immersed in PBS/Tw for 5 minutes in the dark, air dried, covered with a coverslip with fluorescence mounting fluid (Dako) and viewed with a fluorescence microscope. Mock-transfected HEK 293 cells, treated with the same antibodies and substrate, were used as controls.

### (ii) Immunocytochemical Staining

Cells were fixed by adding 2 ml of methanol containing 0.5% H₂O₂ at RT for 15 min. The fixative was removed and replaced with the following monoclonal antibodies diluted in PBS: mAb 19 specific for the F protein diluted 1/400 and mAb 31 specific for the G protein diluted 1/1000. After 1 h incubation at RT, cells were washed ×3 with PBS. HRP goat anti-mouse IgG (Kirkegaard & Perry Laboratories, MD, USA) was diluted 1/2000 in PBS and added to each well. After 1 h incubation at RT, cells were washed x3 with PBS and fresh 3,3-diamino-benzidine (DAB) (Sigma) substrate was made up (10 ml PBS, 0.005 g DAB, 7 μl 30% H₂O₂) and added to each well. After 5 min incubation at RT, or until colour had developed, cells were washed with DDW to stop the reaction. PBS was added to each well and cells were viewed using an inverted microscope to assess the number of stained cells. Mock-transfected cells, treated with the same antibodies and substrate, were used as controls.

(iii) Dot Blot Analysis

The COS-7 cell lysates were thawed and sonicated for 3 min and centrifuged at 14,000×g for 1 min. The supernatant was removed and serial 3-fold dilutions were made in DDW. 100 μl of each dilution was transferred to a nitrocellulose membrane using the Dot Blot™ Apparatus (BioRad, UK). After transfer, the membrane was blocked with 5% skimmed milk in PBS/Tw for 1 h at RT, and then incubated with the appropriate monoclonal antibody, as described previously, diluted 1/2000 in the blocking solution for 1 h. The membrane was washed with PBS/Tw×4 for 5 min and incubated with HRP-conjugated goat anti-mouse IgG diluted 1/20,000 in blocking solution for 1 h. The membrane was washed x4 for 5 min with PBS/Tw and SuperSignal West Pico Chemiluminescent Substrate (Pierce, Ill., USA) was added to the membrane following the manufacturers instructions and incubated at RT for 5 min. Images were developed on film in the dark at several different exposure times. Mock-transfected cells, treated with the same antibodies and substrate, were used as controls. Lysates of RSV-infected vero cells and mock-infected vero cells were used as additional controls.

### (iv) Western Blot Analysis

For Western blot analysis cellular proteins were separated by SDS-polyacrylamide gel electrophoresis (PAGE) under reducing conditions on a 12% gel blotted onto a nitrocellulose membrane. The membrane was blocked overnight in 5% skimmed milk and then the G protein was identified as described above.

### Results

### Production of F and G Codon-Optimised and Wild-Type Plasmids and Analysis of In Vitro Expression

When analysed using the SynGene software, the codon-usage co-efficient for F and G wild-type gene sequences were 0.276 and 0.373 respectively. After codon-optimisation, these were increased to 0.763 for the F gene and 0.753 for the G gene. Plasmids containing codon-optimised or wild type F or G genes were produced as described in the methods section and protein expression was analysed by immunofluorescence. Fluorescent cells were readily detected 24 h after transfection with plasmids encoding the codon-optimised or wild-type G gene (FIG. 1 a & b). In contrast, the number of cells expressing the F protein following transfection with pI.17Fc was lower than that of cells transfected with pI.17Fwt (results not shown).

The G protein was detected in cell lysates from cells transfected with either pGc or pGwt (FIG. 2a). The mature form (Mr of 90 k), an intermediate form (Mr of 46 k) and the unglycosylated form (Mr of 36 k). Significantly more G protein could be detected in supernatants of COS-7 cells transiently transfected with plasmid expressing pGc than those transfected with pGwt.

In order to provide a more quantitative assessment of protein expression, dot blots were produced from cells transfected with plasmids expressing wild-type or codon-optimised F or G. Cell lysates were titrated to provide an indication of the relative amounts of protein produced by transfected cells. Dot blots for codon-optimised and wild-type G showed that expression of G was approximately 9-fold greater in cells transfected with pI.17Gc than those transfected with pI.17 wt (FIG. 2b). In contrast, expression of F from pI.17Fc transfected cells was considerably lower than in cells transfected with pI.17Fwt. (FIGS. 3a and 3b). A four min exposure was required to visualise a faint dot in the lane containing lysate from pI.17Fc transfected cells (FIG. 3a). After 50 min exposure, expression of F by pI.17Fc transfected cells was expressed at least 100-fold lower than that of pI.17Fwt transfected cells. (FIG. 3b).

### APPENDICES

Appendix 1. Wild-type gene sequences

(a) Wild-type F gene sequence

[SEQ ID NO: 3]

| | |
|---|---|
| 1 | ATGGAGTTGC TAATCCTCAA AGCAAATGCA ATTACCACAA TCCTCACTGC |
| 51 | AGTCACATTT TGTTTTGCTT CTGGTCAAAA CATCACTGAA GAATTTTATC |
| 101 | AATCAACATG CAGTGCAGTT AGCAAAGGCT ATCTTAGTGC TCTGAGAACT |
| 151 | GGTTGGTATA CCAGTGTTAT AACTATAGAA TTAAGTAATA TCAAGGAAAA |
| 201 | TAAGTGTAAT GGAACAGATG CTAAGGTAAA ATTGATAAAA CAAGAATTAG |
| 251 | ATAAATATAA AAATGCTGTA ACAGAATTGC AGTTGCTCAT GCAAAGCACA |
| 301 | CCACCAACAA ACAATCGAGC CAGAAGAGAA CTACCAAGGT TTATGAATTA |
| 351 | TACACTCAAC AATGCCAAAA AAACCAATGT AACATTAAGC AAGAAAAGGA |
| 401 | AAAGAAGATT TCTTGGTTTT TTGTTAGGTG TTGGATCTGC AATCGCCAGT |
| 451 | GGCGTTGCTG TATCTAAGGT CCTGCACCTA GAAGGGGAAG TGAACAAGAT |
| 501 | CAAAAGTGCT CTACTATCCA CAAACAAGGC TGTAGTCAGC TTATCAAATG |
| 551 | GAGTTAGTGT CTTAACCAGC AAAGTGTTAG ACCTCAAAAA CTATATAGAT |
| 601 | AAACAATTGT TACCTATTGT GAACAAGCAA AGCTGCAGCA TATCAAATAT |
| 651 | AGAAACTGTG ATAGAGTTCC AACAAAAGAA CAACAGACTA CTAGAGATTA |
| 701 | CCAGGGAATT TAGTGTTAAT GCAGGTGTAA CTACACCTGT AAGCACTTAC |
| 751 | ATGTTAACTA ATAGTGAATT ATTGTCATTA ATCAATGATA TGCCTATAAC |
| 801 | AAATGATCAG AAAAAGTTAA TGTCCAACAA TGTTCAAATA GTTAGACAGC |
| 851 | AAAGTTACTC TATCATGTCC ATAATAAAAG AGGAAGTCTT AGCATATGTA |
| 901 | GTACAATTAC CACTATATGG TGTTATAGAT ACACCCTGTT GGAAACTACA |
| 951 | CACATCCCCT CTATGTACAA CCAACACAAA AGAAGGGTCC AACATCTGTT |
| 1001 | TAACAAGAAC TGACAGAGGA TGGTACTGTG ACAATGCAGG ATCAGTATCT |
| 1051 | TTCTTCCCAC AAGCTGAAAC ATGTAAAGTT CAATCAAATC GAGTATTTTG |
| 1101 | TGACACAATG AACAGTTTAA CATTACCAAG TGAAATAAAT CTCTGCAATG |
| 1151 | TTGACATATT CAACCCCAAA TATGATTGTA AAATTATGAC TTCAAAAACA |
| 1201 | GATGTAAGCA GCTCCGTTAT CACATCTCTA GGAGCCATTG TGTCATGCTA |
| 1251 | TGGCAAAACT AAATGTACAG CATCCAATAA AAATCGTGGA ATCATAAAGA |
| 1301 | CATTTTCTAA CGGGTGCGAT TATGTATCAA ATAAAGGGAT GGACACTGTG |
| 1351 | TCTGTAGGTA ACACATTATA TTATGTAAAT AAGCAAGAAG GTAAAAGTCT |
| 1401 | CTATGTAAAA GGTGAACCAA TAATAAATTT CTATGACCCA TTAGTATTCC |
| 1451 | CCTCTGATGA ATTTGATGCA TCAATATCTC AAGTCAACGA GAAGATTAAC |
| 1501 | CAGAGCCTAG CATTTATTCG TAAATCCGAT GAATTATTAC ATAATGTAAA |
| 1551 | TGCTGGTAAA TCCACCACAA ATATCATGAT AACTACTATA ATTATAGTGA |
| 1601 | TTATAGTAAT ATTGTTATCA TTAATTGCTG TTGGACTGCT CTTATACTGT |
| 1651 | AAGGCCAGAA GCACACCAGT CACACTAAGC AAAGATCAAC TGAGTGGTAT |
| 1701 | AAATAATATT GCATTTAGTA ACTAA |

(b) Wild-type G gene sequence

[SEQ ID NO: 4]

| | |
|---|---|
| 1 | ATGTCCAAAA ACAAGGACCA ACGCACCGCT AAGACATTAG AAAGGACCTG |
| 51 | GGACACTCTC AATCATTTAT TATTCATATC ATCGTGCTTA TATAAGTTAA |
| 101 | ATCTTAAATC TGTAGCACAA ATCACATTAT CCATTCTGGC AATGATAATC |
| 151 | TCAACTTCAC TTATAATTGC AGCCATCATA TTCATAGCCT CGGCAAACCA |
| 201 | CAAAGTCACA CCAACAACTG CAATCATACA AGATGCAACA AGCCAGATCA |
| 251 | AGAACACAAC CCCAACATAC CTCACCCAGA ATCCTCAGCT TGGAATCAGT |
| 301 | CCCTCTAATC CGTCTGAAAT TACATCACAA ATCACCACCA TACTAGCTTC |
| 351 | AACAACACCA GGAGTCAAGT CAACCCTGCA ATCCACAACA GTCAAGACCA |
| 401 | AAAACACAAC AACAACTCAA ACACAACCCA GCAAGCCCAC CACAAAACAA |
| 451 | CGCCAAAACA AACCACCAAG CAAACCCAAT AATGATTTTC ACTTTGAAGT |
| 501 | GTTCAACTTT GTACCCTGCA GCATATGCAG CAACAATCCA ACCTGCTGGG |
| 551 | CTATCTGCAA AAGAATACCA AACAAAAAAC CAGGAAAGAA AACCACTACC |
| 601 | AAGCCCACAA AAAAACCAAC CCTCAAGACA ACCAAAAAAG ATCCCAAACC |
| 651 | TCAAACCACT AAATCAAAGG AAGTACCCAC CACCAAGCCC ACAGAAGAGC |
| 701 | CAACCATCAA CACCACCAAA ACAAACATCA TAACTACACT ACTCACCTCC |
| 751 | AACACCACAG GAAATCCAGA ACTCACAAGT CAAATGGAAA CCTTCCACTC |
| 801 | AACTTCCTCC GAAGGCAATC CAAGCCCTTC TCAAGTCTCT ACAACATCCG |
| 851 | AGTACCCATC ACAACCTTCA TCTCCACCCA ACACACCACG CCAGTAG |

APPENDIX 2
Codon-optimised gene sequences

(a) Codon-optimised F gene sequence

| | |
|---|---|
| | [SEQ ID NO: 5] |
| 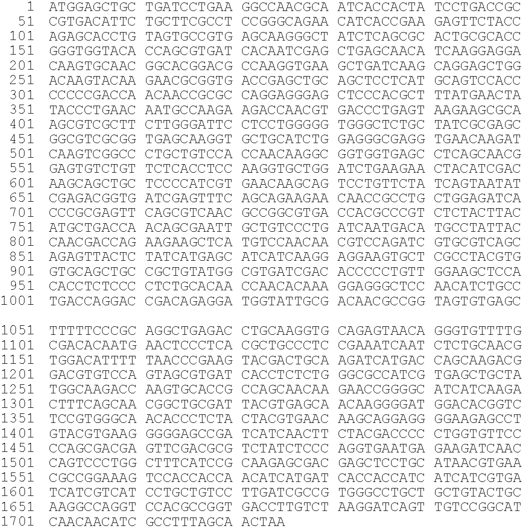 | |

(b) Codon-optimised G gene sequence

| | |
|---|---|
| | [SEQ ID NO: 2] |
| 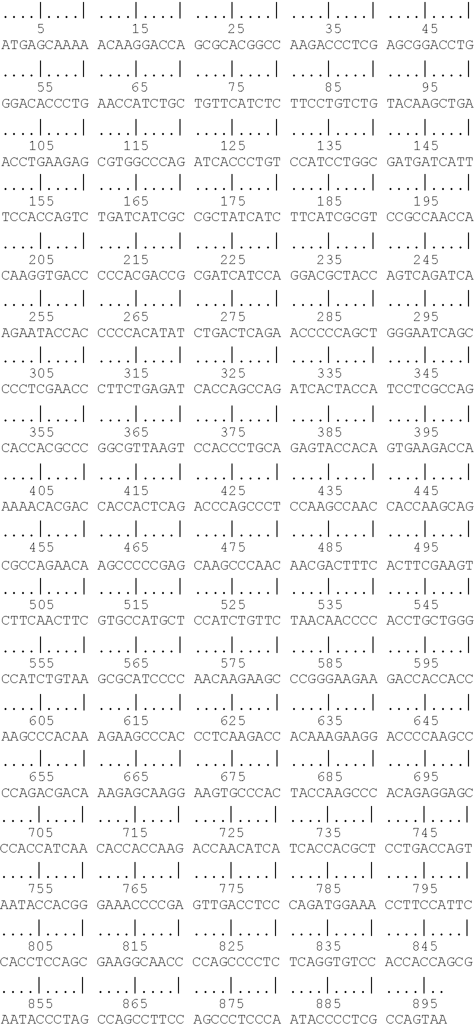 | |

Appendix 3. Oligonucleotides for the production of
synthetic codon-optimised genes by PCR
(u = upper strand, l = lower strand)
(a) Codon-optimised F gene oligonucleotides

| | |
|---|---|
| F-u: 1 | CGCGGATCCAGCATGGAGCTGCTGATCCTGAAGGCCAACGCA |
| | ATCACCACTATCCTGACCGCC |
| | [SEQ ID NO: 6] |
| F-u: 2 | GGCAGAACATCACCGAAGAGTTCTACCAGAGCACCTGTAGTG |
| | CCGTGAGCAAGGGCTATC |
| | [SEQ ID NO: 7] |
| F-u: 3 | GTACACCAGCGTGATCACAATCGAGCTGAGCAACATCAAGGA |
| | GAACAAGTGCAACGGCAC |
| | [SEQ ID NO: 8] |
| F-u: 4 | AAGCAGGAGCTGGACAAGTACAAGAACGCGGTGACCGAGCTG |
| | CAGCTCCTCATGCAGTCC |
| | [SEQ ID NO: 9] |
| F-u: 5 | CCAGGAGGGAGCTCCCACGCTTTATGAACTATACCCTGAACA |
| | ATGCCAAGAAGACCAACG |
| | [SEQ ID NO: 10] |
| F-u: 6 | GCGTCGCTTCTTGGGATTCCTCCTGGGGGTGGGCTCTGCTAT |
| | CGCGAGCGGCGTCGCGGT |
| | [SEQ ID NO: 11] |
| F-u: 7 | GGCGAGGTGAACAAGATCAAGTCGGCCCTGCTGTCCACCAAC |
| | AAGGCGGTGGTGAGCCTC |
| | [SEQ ID NO: 12] |
| F-u: 8 | CCTCCAAGGTGCTGGATCTGAAGAACTACATCGACAAGCAGC |
| | TGCTCCCCATCGTGAACA |
| | [SEQ ID NO: 13] |
| F-u: 9 | TATCGAGACGGTGATCGAGTTTCAGCAGAAGAACAACCGCCT |
| | GCTGGAGATCACCCGCGA |
| | [SEQ ID NO: 14] |
| F-u: 10 | ACCACGCCCGTCTCTACTTACATGCTGACCAACAGCGAATTG |
| | CTGTCCCTGATCAATGAC |
| | [SEQ ID NO: 15] |
| F-u: 11 | AGAAGCTCATGTCCAACAACGTCCAGATCGTGCGTCAGCAGA |
| | GTTACTCTATCATGAGCA |
| | [SEQ ID NO: 16] |
| F-u: 12 | CTACGTGGTGCAGCTGCCGCTGTATGGCGTGATCGACACCCC |
| | CTGTTGGAAGCTCCACAC |
| | [SEQ ID NO: 17] |
| F-u: 13 | ACAAAGGAGGGCTCCAACATCTGCCTGACCAGGACCGACAGA |
| | GGATGGTATTGCGACAAC |
| | [SEQ ID NO: 18] |
| F-u: 14 | CGCAGGCTGAGACCTGCAAGGTGCAGAGTAACAGGGTGTTTT |
| | GCGACACAATGAACTCCC |
| | [SEQ ID NO: 19] |
| F-u: 15 | TCTCTGCAACGTGGACATTTTTAACCCGAAGTACGACTGCAA |
| | GATCATGACCAGCAAGAC |
| | [SEQ ID NO: 20] |
| F-u: 16 | ACCTCTCTGGGCGCCATCGTGAGCTGCTATGGCAAGACCAAG |
| | TGCACCGCCAGCAACAAG |
| | [SEQ ID NO: 21] |
| F-u: 17 | TCAGCAACGGCTGCGATTACGTGAGCAACAAGGGGATGGACA |
| | CGGTCTCCGTGGGCAACA |
| | [SEQ ID NO: 22] |
| F-u: 18 | GGAGGGGAAGAGCCTGTACGTGAAGGGGGAGCCGATCATCAA |
| | CTTCTACGACCCCCTGGT |
| | [SEQ ID NO: 23] |
| F-u: 19 | GCGTCTATCTCCCAGGTGAATGAGAAGATCAACCAGTCCCTG |
| | GCTTTCATCCGCAAGAGC |
| | [SEQ ID NO: 24] |
| F-u: 20 | ACGCCGGAAAGTCCACCACCAACATCATGATCACCACCATCA |
| | TCATCGTGATCATCGTCA |
| | [SEQ ID NO: 25] |
| F-u: 21 | GGGCCTGCTGCTGTACTGCAAGGCCAGGTCCACGCCGGTGAC |
| | CTTGTCTAAGGATCAGTT |
| | [SEQ ID NO: 26] |
| F-1: 1 | CCGGAATTCTTAGTTGCTAAAGGCGATGTTGTTGATGCCGGA |
| | CAACTGATCCTTAGACAAGG |
| | [SEQ ID NO: 27] |
| F-1: 2 | GCAGTACAGCAGCAGGCCCACGGCGATCAAGGACAGCAGGAT |
| | GACGATGATCACGATGAT |
| | [SEQ ID NO: 28] |
| F-1: 3 | GTGGTGGACTTTCCGGCGTTCACGTTATGCAGGAGCTCGTCG |
| | CTCTTGCGGATGAAAGCC |
| | [SEQ ID NO: 29] |
| F-1: 4 | TCACCTGGGAGATAGACGCGTCGAACTCGTCGCTGGGGAACA |
| | CCAGGGGGTCGTAGAAGT |
| | [SEQ ID NO: 30] |
| F-1: 5 | GTACAGGCTCTTCCCCTCCTGCTTGTTCACGTAGTAGAGGGT |
| | GTTGCCCACGGAGACCGT |
| | [SEQ ID NO: 31] |
| F-1: 6 | TAATCGCAGCCGTTGCTGAAAGTCTTGATGATGCCCCGGTTC |
| | TTGTTGCTGGCGGTGCAC |
| | [SEQ ID NO: 32] |
| F-1: 7 | CGATGGCGCCCAGAGAGGTGATCACGCTACTGGACACGTCCG |
| | TCTTGCTGGTCATGATCT |
| | [SEQ ID NO: 33] |
| F-1: 8 | AATGTCCACGTTGCAGAGATTGATTTCGGAGGGCAGCGTGAG |
| | GGAGTTCATTGTGTCGCA |
| | [SEQ ID NO: 34] |
| F-1: 9 | TTGCAGGTCTCAGCCTGCGGGAAAAAGCTCACACTACCGGCG |
| | TTGTCGCAATACCATCCT |
| | [SEQ ID NO: 35] |
| F-1: 10 | TGTTGGAGCCCTCCTTTGTGTTGGTTGTGCAGAGGGGAGAGG |
| | TGTGGAGCTTCCAACAGG |
| | [SEQ ID NO: 36] |
| F-1: 11 | CGGCAGCTGCACCACGTAGGCGAGCACTTCCTCCTTGATGAT |
| | GCTCATGATAGAGTAACT |
| | [SEQ ID NO: 37] |
| F-1: 12 | TTGTTGGACATGAGCTTCTTCTGGTCGTTGGTAATAGGCATG |
| | TCATTGATCAGGGACAGC |
| | [SEQ ID NO: 38] |
| F-1: 13 | AAGTAGAGACGGGCGTGGTCACGCCGGCGTTGACGCTGAACT |
| | CGCGGGTGATCTCCAGCA |
| | [SEQ ID NO: 39] |
| F-1: 14 | CTCGATCACCGTCTCGATATTACTGATAGAACAGGACTGCTT |
| | GTTCACGATGGGGAGCAG |
| | [SEQ ID NO: 40] |
| F-1: 15 | AGATCCAGCACCTTGGAGGTGAGAACAGACACTCCGTTGCTG |
| | AGGCTCACCACCGCCTTG |
| | [SEQ ID NO: 41] |
| F-1: 16 | TGATCTTGTTCACCTCGCCCTCCAGATGCAGCACCTTGCTCA |
| | CCGCGACGCCGCTCGCGA |
| | [SEQ ID NO: 42] |
| F-1: 17 | GAATCCCAAGAAGCGACGCTTGCGCTTCTTACTCAGGGTCAC |
| | GTTGGTCTTCTTGGCATT |
| | [SEQ ID NO: 43] |
| F-1: 18 | CGTGGGAGCTCCCTCCTGGCGCGGTTGTTGGTCGGGGGGGTG |
| | GACTGCATGAGGAGCTGC |
| | [SEQ ID NO: 44] |
| F-1: 19 | ACTTGTCCAGCTCCTGCTTGATCAGCTTCACCTTGGCGTCCG |
| | TGCCGTTGCACTTGTTCT |
| | [SEQ ID NO: 45] |
| F-1: 20 | TGTGATCACGCTGGTGTACCACCCGGTGCGCAGTGCGCTGAG |
| | ATAGCCCTTGCTCACGGC |
| | [SEQ ID NO: 446] |
| F-1: 21 | TCTTCGGTGATGTTCTGCCCGGAGGCGAAGCAGAATGTCACG |
| | GCGGTCAGGATAGTGGTG |
| | [SEQ ID NO: 47] |

(b) Codon-optimized G gene oligonucleotides

| | |
|---|---|
| G-u: 1 | CGCGGATCCAGCATGAGCAAAAACAAGGACCAGCGCACGGCC |
| | AAGACCCTCGAGCGGAC |
| | [SEQ ID NO: 48] |
| G-u: 2 | CTGTTCATCTCTTCCTGTCTGTACAAGCTGAACCTGAAGAGC |
| | GTGGCCCAGATCACCCTG |
| | [SEQ ID NO: 49] |
| G-u: 3 | CCACCAGTCTGATCATCGCCGCTATCATCTTCATCGCGTCCG |
| | CCAACCACAAGGTGACCC |
| | [SEQ ID NO: 50] |
| G-u: 4 | CGCTACCAGTCAGATCAAGAATACCACCCCCACATATCTGAC |
| | TCAGAACCCCCAGCTGGG |
| | [SEQ ID NO: 51] |
| G-u: 5 | GAGATCACCAGCCAGATCACTACCATCCTCGCCAGCACCACG |
| | CCCGGCGTTAAGTCCACC |
| | [SEQ ID NO: 52] |
| G-u: 6 | CCAAAAACACGACCACCACTCAGACCCAGCCCTCCAAGCCAA |
| | CCACCAAGCAGCGCCAGA |
| | [SEQ ID NO: 53] |
| G-u: 7 | CAACGACTTTCACTTCGAAGTCTTCAACTTCGTGCCATGCTC |
| | CATCTGTTCTAACAACCC |
| | [SEQ ID NO: 54] |
| G-u: 8 | CGCATCCCCAACAAGAAGCCCGGGAAGAAGACCACCACCAAG |
| | CCCACAAAGAAGCCCACC |
| | [SEQ ID NO: 55] |
| G-u: 9 | CCAAGCCCCAGACGACAAAGAGCAAGGAAGTGCCCACTACCA |
| | AGCCCACAGAGGAGCCCA |
| | [SEQ ID NO: 56] |
| G-u: 10 | CATCATCACCACGCTCCTGACCAGTAATACCACGGGAAACCC |
| | CGAGTTGACCTCCCAGAT |
| | [SEQ ID NO: 57] |
| G-u: 11 | AGCGAAGGCAACCCCAGCCCCTCTCAGGTGTCCACCACCAGC |
| | GAATACCCTAGCCAGCCT |
| | [SEQ ID NO: 58] |
| G-1: 1 | CCGGAATTCTTACTGGCGAGGGGTATTGGGAGGGCTGGAAGG |
| | CTGGCTAGGGTATTCG |
| | [SEQ ID NO: 59] |
| G-1: 2 | GGCTGGGGTTGCCTTCGCTGGAGGTGGAATGGAAGGTTTCCA |
| | TCTGGGAGGTCAACTCGG |
| | [SEQ ID NO: 60] |
| G-1: 3 | CAGGAGCGTGGTGATGATGTTGGTCTTGGTGGTGTTGATGGT |
| | GGGCTCCTCTGTGGGCTT |
| | [SEQ ID NO: 61] |
| G-1: 4 | TTTGTCGTCTGGGGCTTGGGGTCCTTCTTTGTGGTCTTGAGG |
| | GTGGGCTTCTTTGTGGGC |
| | [SEQ ID NO: 62] |
| G-1: 5 | GCTTCTTGTTGGGGATGCGCTTACAGATGGCCCAGCAGGTGG |
| | GGTTGTTAGAACAGATGG |
| | [SEQ ID NO: 63] |
| G-1: 6 | TTCGAAGTGAAAGTCGTTGTTGGGCTTGCTCGGGGGCTTGTT |
| | CTGGCGCTGCTTGGTGGT |
| | [SEQ ID NO: 64] |
| G-1: 7 | GTGGTGGTCGTGTTTTTGGTCTTCACTGTGGTACTCTGCAGG |
| | GTGGACTTAACGCCGGGC |
| | [SEQ ID NO: 65] |
| G-1: 8 | TGATCTGGCTGGTGATCTCAGAAGGGTTCGAGGGGCTGATTC |
| | CCAGCTGGGGGTTCTGAG |
| | [SEQ ID NO: 66] |
| G-1: 9 | CTTGATCTGACTGGTAGCGTCCTGGATGATCGCGGTCGTGGG |
| | GGTCACCTTGTGGTTGGC |
| | [SEQ ID NO: 67] |
| G-1: 10 | GCGATGATCAGACTGGTGGAAATGATCATCGCCAGGATGGAC |
| | AGGGTGATCTGGGCCACG |
| | [SEQ ID NO: 68] |
| G-1: 11 | GACAGGAAGAGATGAACAGCAGATGGTTCAGGGTGTCCCAGG |
| | TCCGCTCGAGGGTCTTGG |
| | [SEQ ID NO: 69] |

Appendix 4. PCR Assembly Reaction to Produce a Gene Product from Synthetic Oligonucleotides

The following reaction was set up for each oligonucleotide pool:

5 μl 10× buffer (Roche)
1 μl oligonucleotide pool (equal volumes of each oligonucleotide at a dilution of 100 pmol/μl)
1 μl 25 mM dNTPs
42 μl water
5 U PWO polymerase (Roche)

The following PCR cycle was programmed into a PCR block:

### (1). 94° C. for 30 sec

### (2). 45° C. for 120 sec

### (3). 72° C. for 10 sec

### (4). 94° C. for 15 sec

### (5). 45° C. for 15 sec

(6). 72° C. for 20 sec+3 sec per cycle−cycle to step (4) 25 times
(7). 4° C. hold
Appendix 5. PCR Recovery Reaction to Amplify the Desired Full Length Gene Product from the Assembly Reaction

The following was set up for each assembly reaction:

4 μl 10× buffer (Roche)
2 μl assembly reaction
0.25 μl of each end primer
1 μl 25 mM dNTPs
40 μl water
5 U PWO polymerase (Roche)

The following PCR cycle was programmed into a PCR block:

### (1). 94° C. for 45 sec

(2). 72° C. for 120 sec−cycle to step (1) 25 times

### (3). 72° C. for 240 sec

(4). 4° C. hold

### Appendix 6. Restriction Reaction

The following restriction reactions were set up:

25 μl PCR product
5 μl buffer 3 (Invitrogen)

### 2 μl EcoR1 (Invitrogen)

### 2 μl BamH1 (Invitrogen)

16 μl water
0.3 μl pI.17 (4 nm/μl)
5 μl buffer 3 (Invitrogen)

### 2 μl EcoR1 (Invitrogen)

### 2 μl BamH1 (Invitrogen)

16 μl water

Reactions were incubated at 37° C. for 2 h and then held at 4° C.

### Appendix 7. Ligation Reaction

1 μl pI.17 vector cut with EcoR1 and BamH1
1 μl PCR product cut with EcoR1 and BamH1
2 μl water
5 μl 2× buffer (Promega)
1 μl ligase (Promega)

Incubated at room temperature for 15 min.

### Appendix 8. Sequencing of Plasmid Inserts

96-well plates were set up with each well containing 1.0 ug Plasmid DNA and 5.0 pmol primer for each reaction required, made up to 10 ul in water and sent to be sequenced. Results were then analysed using the SeqMan software.

The following reactions were set up and then run on the sequencing machine:
1. 0.5 ml PCR tubes set up for each reaction containing 300 ng plasmid DNA made up to 5 μl in water.
2. Heated to 96° C. for 1 minute
3. 5.0 pmol of primer added to tube
4. 4 μl Quickstart mastermix (Beckman Coulter) added to each tube
5. Tubes placed in PCR block and the following cycle was run:
   (1) 96° C. for 20 sec
   (2) 50° C. for 20 sec 30 cycles
   (3) 60° C. for 4 min
   (4) 4° C. hold
6. Reactions transferred to clean tubes.
7. 5 μl Stop solution added to each tube (40% 100 mM EDTA (Sigma), 40% 3 M NaOAc (Sigma), 20% glycogen (Roche))
8. 60 μl ice-cold 95% ethanol added to each sample and vortexed to mix.
9. Centifuged at 13,000×g for 15 mins at 4° C.
10. Supernatent removed.
11. 200 μl ice-cold 70% ethanol added
12. Centifuged at 13,000×g for 5 minutes at 4° C.
13. Supernatent removed.
14. Steps 11-13 repeated. All ethanol removed.
15. Pellet air-dried for at least 1 hour.
16. Pellet resuspended in 40 μl sample loading solution (Beckman Coulter). Left for 1 min and then vortexed.
17. 1 drop of mineral oil added to each tube and samples frozen at −20° C. overnight.
18. Samples run on capillary sequencer and results analysed using the GCG software.

### Appendix 9. Amplification of Correct Gene Fragments

The following reaction was set up for each correct gene fragment:

5 μl 10× buffer (Roche)
0.5 μl mini-prep
0.25 μl each primer (1 forward and 1 reverse primer to amplify only the correct fragment)
1 μl dNTPs
43 μl water
0.5 μl PWO polymerase (Roche)

The following PCR cycle was programmed into a PCR block:

### (1). 95° C. for 30 s

### (2). 95° C. for 30 s

### (3). 50° C. for 30 s

(4). 72° C. for 90 s (˜2 min/kb)
(5). Cycle to step (2) 20 times

### (6). 72° C. for 120 s

(7). 4° C. hold

### Appendix 10. Stitching Together Amplified Fragments by PCR

The following reaction was set up to stitch all amplified fragments for each gene together:

5 μl 10× buffer (Roche)
2 μl each amplified fragment
0.25 μl of each end primer (lowest numbered upper and lower oligonucleotides)
1 μl dNTPs
0.5 μl PWo polymerase (Roche)
water to make mixture up to 50 μl

The following PCR cycle was programmed into a PCR block:

### (1). 95° C. for 30 s

### (2). 95° C. for 30 s

### (3). 50° C. for 30 s

(4). 72° C. for 90 s (˜2 min/kb)
(5). Cycle to step (2) 20 times

### (6). 72° C. for 120 s

(7). 4° C. hold
Appendix 11. Wild-Type Oligonucleotides Used to Amplify the Wild-Type Genes from GSK Plasmids and Add Restriction Sites to the Front and Back of the Genes (u=Upper Strand, l=Lower Strand)

| | |
|---|---|
| FU1 | |
| CGCGGATCCAGCATGGAGTTGCTAATCCTCAA | [SEQ ID NO: 70] |
| FL1 | |
| GGCGAATTCTTAGTTACTAAATGCAATAT | [SEQ ID NO: 71] |
| GU1 | |
| CGCGGATCCAGCATGTCCAAAAACAAGGACCA | [SEQ ID NO: 72] |
| GL1 | |
| GGCGAATTCCTACTGGCGTGGTGTGTTGG | [SEQ ID NO: 73] |

### Example B

### Immunoprotective Effect of Codon-Optimised G

### Methods

See Example A for detailed protocols.

Empty pI.17 plasmid (pCon), was used as a control plasmid.

### Gene Gun Cartridge Preparation

DNA was precipitated onto 1.6 μm gold beads (BioRad, UK) following the supplier's guidelines. Briefly, 100 μl 1M CaCl₂ was added to 25 mg of gold in 100 μl of 0.05M spermidine (Sigma-Aldrich) and 100 μg DNA. Following precipitation, the gold beads were washed extensively with dry ethanol before being re-suspended in 3 ml 0.05 mg ml⁻¹ polyvinylpyrrolidone (PVP) (BioRad) in dry ethanol and loaded into Tefzel tubing (BioRad). Ethanol was drawn off and the gold dried onto the surface of the tubing with N2 gas. Each cartridge contained 0.5 mg gold and 1.5 μg DNA. Gold only cartridges were made following the same procedure but omitting the DNA.

### Virus

Stocks of the A2 strain of HRSV were grown in foetal calf kidney (FCK) cells as described previously (see Stott et al., 1984, J Hyg (Loud) 93(2):251-61). Virus pools containing 10⁷ pfu ml⁻¹ were stored in liquid N2 and used in all experiments.

### Animals and Experimental Design

Six-week-old, specific-pathogen-free, female BALB/c mice were obtained from Charles River (UK). Mice were vaccinated using the Helios Gene Gun™ (BioRad, UK) with a single shot of 1.5 μg of plasmid at 300 psi to freshly shaven skin on the abdomen (Bembridge et al., 2000, Journal of General Virology 81(Pt 10):2519-2523) either once or twice 3 weeks apart.

Mice were challenged intranasally (i.n.) 3 weeks after the final vaccination with 10⁵ pfu of RSV in a volume of 50 μl as described previously (see Taylor et al., 1984, Infect Immun 43(2):649-55). Mice were killed either 5 days after challenge with an overdose of sodium pentobarbitone and RSV titres in lung homogenates were determined by plaque assay on FCK cells as described previously (see Taylor et al., 1984, Infect Immun 43(2):649-55), 6 days after challenge and bronchoalveolar lavage (BAL) was obtained using 1 ml of 12 mM lidocaine as described previously (see Bembridge et al., 2000, Journal of General Virology 81(Pt 10):2519-2523) or were killed 3 weeks after challenge. All mice were test bled at intervals for serology.

### Serology

Antibody responses to RSV were determined by enzyme-linked immunosorbent assay (ELISA) as described previously (see Stott et al., 1987, J Virol 61(12):3855-61). A lysate of HRSV-infected vero cells was used as antigen, whilst a lysate of mock-infected cells was used as control antigen. Bound antibody was detected using HRP-conjugated goat anti-mouse IgG (Kirkegaard & Perry Laboratories, MD, USA) or HRP-conjugated rat anti-mouse IgG1 or IgG2a (PharMingen, San Diego, Calif.) for isotype-specific ELISA.

The ability of the serum antibody to neutralise RSV was determined by a plaque reduction assay as described previously (see Kennedy et al., 1988, Journal of General Virology 69(12):3023-3032). Dilutions of pooled serum (150 μl) were mixed with 150 μl of Jacobs 7.8 medium containing 50 p.f.u. RSV and incubated at room temperature for 1 h. Medium containing virus but no antibody was used as a control. The virus and antibody mixtures were then inoculated onto FCK cells. Following an absorption period of 1 h, the virus-antibody mixtures were removed and the cell sheet overlaid with 2 ml medium containing 0.25% agarose. Plates were incubated at 37° C. for 7 days and then fixed with 10% formal-saline and stained with crystal violet.

### Results

### Immunogenicity of Codon-Optimised and Wild-Type G In Vivo

As expression of the G protein was increased in vitro by codon-optimisation of the RSV-G sequence, we speculated that the immune response in mice vaccinated by gene gun with the pGc would be greater than that induced by vaccination with pGwt. First we looked at the effect of codon-optimisation on humoral immune responses.

BALB/c mice were vaccinated by gene gun either once or on 2 occasions, 3 weeks apart, with pGc, pGwt or pCon and test bled at weekly intervals.

Vaccination with either pGc or pGwt induced substantial RSV-specific ELISA antibody responses (FIG. 4b). However, mice vaccinated with pGc showed a more rapid induction of RSV-specific serum antibody compared to those vaccinated with pGwt (FIG. 4a). 6 weeks after vaccination the predominant antibody isotype was IgG1 (FIG. 4b), suggesting a Th2-type immune response typical of gene gun vaccination. There were no significant differences in the ELISA antibody titres between mice vaccinated with pGc or pGwt at this time point. However, the neutralising antibody titre at the time of challenge was greater in mice vaccinated twice with pGc (log₁₀ titre=0.9) than in mice vaccinated twice with pGwt (log₁₀ titre=<0.5).

In order to determine if codon-optimisation of the G protein enhanced protective efficacy, mice were vaccinated by gene gun as above and challenged i.n. with RSV 3 weeks after the second immunization. Immunisation with 2 doses of pGc induced almost complete protection against RSV challenge (FIG. 5). Virus titres were significantly lower in mice vaccinated with 2 doses of pGc than in those vaccinated with 2 doses of pGwt (p<0.05), demonstrating that codon-optimisation of the G protein enhanced protection against RSV challenge.

Duration of Immunity Induced by DNA Vaccination with Codon-Optimised G (pGc)

Codon-optimisation of the RSV-G sequence resulted in improved protein expression and improved protection against RSV infection. The duration of immunity induced by gene-gun vaccination with pGc was compared with that induced by a plasmid expressing the wild-type F.

Mice were vaccinated using the gene gun on 2 occasions, 3 weeks apart with 1.5 μg pGc, pFwt, pCon or were not vaccinated. Mice were test bled at 2-monthly intervals and RSV-specific antibody was analysed by ELISA. High levels of RSV-specific serum antibodies were detected in mice vaccinated with either pGc or pFwt and were maintained at a similar level for 6 months (FIG. 6).

Values shown are the mean log 10 RSV specific antibody titres±s.d. (n=5). IgG titres were significantly higher in mice vaccinated with pGc than in those vaccinated with pFwt at 4 months (p<0.05) and 6 months (p<0.03).

Mice were challenged i.n. with RSV at either 2, 4 or 6 months post vaccination. Virus titres in lung homogenates were analysed 5 days after infection by plaque assay. Values shown in FIG. 7 are the mean titre (log 10 pfu/ml) of RSV in lung homogenates±s.d. (n=5). Vaccination with either pGc or pFwt resulted in a statistically significant reduction in virus titre compared to control mice at 2, 4 and 6 months post vaccination (FIG. 7). At all time points after vaccination, protection conferred by pGc was greater than that conferred by pFwt. Thus, at 2 months post vaccination RSV was isolated in low titres from lungs of 4 out of 5 mice vaccinated with pFwt and only 1 out of 5 mice vaccinated with pGc. At 4 and 6 months after vaccination RSV was isolated from the lungs of all mice vaccinated with pFwt and from only 3 out of 5 mice vaccinated with pGc.

Differences in virus titre between mice vaccinated with pFwt or pGc were only statistically significant at 6 months (p<0.02). By 6 months post vaccination, mice vaccinated with pFwt had a 100-fold reduction in virus titre, compared to control mice, whereas there was a 1000-fold reduction in virus titre in mice vaccinated with pGc.

1. An isolated polynucleotide comprising a nucleotide sequence encoding the G protein of human respiratory syncytial virus (RSV), wherein the nucleotide sequence is codon optimised for expression in mammalian cells and wherein the polynucleotide provides increased expression of the G protein in mammalian cells relative to expression of the wildtype RSV-G gene in the same mammalian cell.

2. A polynucleotide according to claim 1 wherein the mammalian cells are human cells.

3. A polynucleotide according to claim 2 wherein the human cells are HEK 293 cells.

4. A polynucleotide according to claim 1 wherein expression of the codon-optimised polynucleotide in mammalian cells is increased by at least 100% compared to expression of the wildtype RSV-G gene.

5. A polynucleotide according to claim 4 comprising the nucleotide sequence of SEQ ID NO:2.

6. A polynucleotide according to claim 5 consisting of the nucleotide sequence of SEQ ID NO:2.

7. A vector comprising a polynucleotide according to claim 6.

8. A vector according to claim 7 wherein the vector is an expression vector.

9. A vector according to claim 7 selected from the group consisting of PIV-3, Sendai virus, vaccinia virus, fowlpox virus, respiratory syncytial virus and adenovirus.

10. A vector according to claim 9 wherein the vector is pI.17.

11. A mammalian host cell comprising a polynucleotide according to claim 6, optionally being cloned in a vector.

12. A host cell according to claim 11 selected from the group consisting of human embryonic kidney cells, Chinese hamster ovary cells, NIH Swiss mouse embryo cells NIH/3T3, and monkey kidney-derived COS-1 cells.

13. A host cell according to claim 12 wherein the host cell is an HEK 293 cell.

14. A method for producing protein, or immunogenic fragment or variant thereof, the method comprising expressing a polynucleotide according to claim 1 in a host cell, and isolating the expressed polypeptide therefrom.

15. A method according to claim 14 wherein the G protein, or immunogenic fragment or variant thereof is a polypeptide according to SEQ ID NO:1.

16. A method according to claim 15 further comprising admixing the expressed polypeptide with a pharmaceutically acceptable excipient, diluent or carrier to produce a pharmaceutical composition.

17. A method according to claim 16 wherein the pharmaceutical composition is a vaccine composition.

18. A pharmaceutical composition comprising a polynucleotide according to claim 6 and a pharmaceutically acceptable excipient, diluent or carrier.

19. A pharmaceutical composition according to claim 18 wherein the composition is a vaccine composition.

20. A method for producing a pharmaceutical composition according to claim 18, the method comprising admixing said polynucleotide with a pharmaceutically acceptable excipient, diluent or carrier.

21. A method of immunising a subject against infection with respiratory syncytial virus, the method comprising administering to the subject a pharmaceutical composition according to claim 19.

22-30. (canceled)
